(19) United States
(12) Patent Application Publication
(10) Pub. No.: US 2017/0283762 A1
(43) Pub. Date: Oct. 5, 2017

(54) METHOD OF ISOLATING PROBIOTICS OF HUMAN INTESTINE

(71) Applicants: Guangzhou Kangze Medical Technology Co., Ltd., Guangdong (CN); Zhujiang Hospital of Southern Medical University, Guangdong (CN)

(72) Inventors: Yongzheng PENG, Guangdong (CN); Jiangfeng YI, Guangdong (CN); Xianfeng GUO, Guangdong (CN)

(21) Appl. No.: 15/510,238

(22) PCT Filed: Nov. 23, 2015
(86) PCT No.: PCT/CN2015/000809

§ 371 Date: Mar. 10, 2017

(30) Foreign Application Priority Data
Oct. 23, 2015 (CN) 201510706483.1

(51) Int. Cl. C12N 1/02

(52) U.S. Cl. CPC C12N 1/02

(57) ABSTRACT

A method of isolating probiotics of human intestine, including preparing bases for culturing liquid and solid mucoproteins; and filtering, which includes collecting human feces, planting the human feces in the base for culturing liquid mucoprotein, culturing the human feces to obtain microbial liquid, using the microbial liquid as template, pouring the microbial liquid into tubes, subjecting the tubes to a PCR test, selecting the tubes tested positive in the PCR test, pouring the microbial liquid in the positive tubes into the base for culturing solid mucoprotein, culturing the microbial liquid to obtain first colonies, planting the colonies in a chocolate tablet, culturing the first colonies to obtain second colonies, planting the second colonies in a Columbia blood tablet, purifying the second colonies to obtain third colonies, using 16sRNA common primers to identify the third colonies in the PCR, and isolating probiotics.

### BACKGROUND OF THE INVENTION

The invention relates to isolation of microorganisms of human intestine, and more particularly relates to a method of isolating probiotics of human intestine.

Probiotics are microorganisms that are believed to provide health benefits when consumed. However, conventions i methods of isolating and purifying probiotics in the field of microorganisms are not developed and not efficient. There was a probiotic C-IFH strain named Akkemiansia muciniphila isolated in Europe in 2004. C-IFH has a plantation percentage of about 100% in European adults and is capable of inducing and supporting weight loss and sugar reduction.

However, conventional methods of isolating C-IFH strains are complicated and expensive. No further probiotic strains related to C-IFH are isolated and reported. How to provide a novel method of isolating and purifying probiotics related to C-IFH strains is thus desirable for the technical breakthrough concerning the correlation between C-IFG strains and associated diseases.

### BRIEF SUMMARY OF THE INVENTION

The invention has been made in an effort to solve the problems of the prior art including slow isolation of probiotics.

It is therefore an object of the invention to provide a method of isolating probiotics of human intestine, comprising the following steps:

Step 1: preparing culture bases, including preparing a base for culturing liquid mucoprotein and preparing a base for culturing solid mucoprotein; and

Step 2: filtering, including collecting human feces samples, planting the human feces samples in the base for culturing liquid mucoprotein, culturing the human feces samples in an anaerobic environment to obtain microbial liquid, using the microbial liquid as template for PCR test which comprises pouring the microbial liquid into tubes and subjecting the tubes to PCR test, selecting the tubes tested positive in PCR test, planting the microbial liquid in the tubes tested positive on the base for culturing solid mucoprotein, culturing the microbial liquid on the base for culturing solid mucoprotein in an anaerobic environment to obtain first colonies, planting the first colonies in a chocolate tablet, culturing the, first colonies in an anaerobic environment to obtain second colonies, planting the second colonies in a Columbia blood tablet, purifying the second colonies in an anaerobic environment to obtain third colonies, using 16sRNA common primers to identify the third colonies in PCR sequence identification, and isolating probiotics of human intestine.

Preferably, the mucoprotein in both the base for culturing solid mucoprotein and the base for culturing liquid mucoprotein is purified mucoprotein.

Preferably, the primers used in the PCR test are primer 1 having the sequence of SEQ ID NO: 1 (CAGCACGTGAAGGTGGGGAC), and primer 2 having the sequence of SEQ ID NO: 2 (CCTTGCGGTTGGCTTCAGAT).

Preferably, the human feces samples are planted on the base for culturing liquid mucoprotein for culturing in an anaerobic environment at 37° C. for four days,

Preferably, the microbial liquid is planted on the base for culturing solid mucoprotein for culturing in an anaerobic environment at 37° C. for three days,

Preferably, the first colonies are planted on a chocolate tablet for culturing in anaerobic environment at 27° C. for two days.

Preferably, the planting of the second colonies in a Columbia blood tablet for purification is repeated three times, wherein in each time, purification temperature is set at 37° C. and lasts for five days.

Purification of the mucoprotein comprises the following steps:

Reagents preparation:

Ethanol without water (cooled at 4° C.)

Solution A: 0.2 g of NaH₂PO₄ without water, 7 g of Na₂HPO₄·12H₂O, 6 g of NaCL, pH adjusted to 7.8, constant volume being adjusted to 1,000 ml, kept at high pressure and 125° C. for 30 mins, and subsequently kept at room temperature;

Solution B: 5.85 g of NaCL, pH adjusted to 7.0, constant volume being adjusted to 1,000 ml, and kept at high pressure and 125° C. for 30 mins; and subsequently kept at room temperature.

10-15 g of mucoprotein powder is added to a reagent bottle containing 500 ml of solution A to form a solution. The solution is agitated evenly by a magnetic agitator for hours. 1M sodium hydroxide is added to the solution to adjust its pH value to 7.2±0.2. 500-1,000 μl of toluene is added to the solution. The solution is then evenly agitated again by the magnetic agitator for 18 hours. Next,the solution is rotated by a centrifugal device rotating at 3,000 rpm for 10 mins. Liquid upper portion of the solution is collected and transferred to a second reagent bottle which has been subjected to high pressure, and deposited residue of the solution is discarded. Cooled ethanol without water is added to the liquid upper portion in the second reagent bottle to reach a concentration percentage of about 60%. The second reagent bottle: placed in a refrigerator and kept at 4° C. for 30 mins. The second reagent bottle Is rotated by the centrifugal device rotating at 3,000 rpm for 10 mins to obtain a second liquid upper portion and a second deposited residue. The second d upper portion is discarded and the second deposited residue is collected. The second deposited residue is dissolved in 200 ml of solution B, and then agitated for 6 hours, and after that rotated by the centrifugal device rotating at 3,000 rpm for 10 mins to obtain a third liquid upper portion and a third deposited residue. The third liquid upper portion is transferred to a third reagent bottle which has been subjected to high pressure, and the third deposited residue is discarded. Cooled ethanol without water is added to the third liquid upper portion in the third reagent bottle to reach a concentration percentage of about 60%. The third reagent bottle is placed in a refrigerator and kept at 4° C. for 30 mins. After that, the third reagent bottle is rotated by the centrifugal device rotating at 3,000 rpm for 10 mins to obtain a fourth liquid upper portion and a fourth deposited residue. The fourth liquid upper portion is discarded and the fourth deposited residue is collected. The fourth deposited residue is dissolved in 100 ml of distilled water and kept in a sealed condition at 4° C.

Preferably, the base for culturing liquid mucoprotein is prepared as follows:

Reagents preparation:

Add chemical compounds, including 1.491 g of FeCL₂·4H₂O, 0.06 g of H₃BO₄, 0.068 g of ZnCL₂, 0.1725 g of CuCL·7H₂O, 0.0635 g of MnCL₂, 0.0119 g of CoCL₂·6H₂O, 0.0235 g of NiCL₂·6H₂O, 4.18 ml of 37% HCL, and the rest being distilled water to make up a constant volume of 100 ml.

Basic chemical compounds, including 0.01729 g of Na₂SeO₃, 0.0242 g of Na₂MoO₄·2H₂O, 0.4 g of NaOH, and the rest being distilled water to make up a constant volume of 100 ml.

Liquid vitamin, including 0.02 g of biotin, 0.21 g of Vit B₃, 0.5 g of Vit B₆, 0.1 g of Vit B₂, 0.2 g of Vit B₁, 0.25 g of Vit B₁₂, 0.1 g of pantothenic acid, and the rest being distilled water to make up a constant volume of 100 ml.

Solution 1, including 1.1 g of CaCL₂, 1.0 g of MgCL₂, 1 ml of the add chemical con pounds, 1 ml of the basic chemical compounds, and the rest being distilled water to make up a constant volume of 100 ml; said solution 1 is kept at high pressure and 125° C. for 30 mins and is subsequently kept at room temperature in a sealed condition.

Solution 2, including 5.3 g of Na₂HPO₄, 3 g of NaCL, 4 g of KH₂PO₄, and the rest being distilled water to make up a constant volume of 100 ml; said solution 2 is kept at high pressure and 125° C. for 30 mins, and is subsequently kept at room temperature in a sealed condition.

Solution 3, including 2 g of NaHCO₃, 2 ml of the liquid vitamin, constant volume being adjusted to 100 ml, filtered by a 0.22 μm filter, and is subsequently kept at 4° C. in a sealed condition.

Solution 4, including 2.5 g of Na₂S and the rest being distilled water to make up a constant volume of 100 ml, filtered by 0.22 μm filter, and is subsequently kept at 4° C. in a sealed condition.

Adding 200 ml of sterilized distilled water to beaker that has been subjected to high pressure, sequentially adding 2 ml of solution 1, 2 ml of solution 2, 1 ml of solution 3, 2 ml of solution 4 and 30-40 ml of purified mucoprotein solution to the beaker on a clean bench to form a liquid mixture wherein the beaker is shaken evenly after each adding step, sucking 5 ml of the liquid mixture by means of an electric liquid suction device and adding 5 ml of the liquid mixture to each of a plurality of tubes having 15 ml volume, sealing the tubes, and keeping the tubes at 4° C. in a sealed condition.

Preferably, the base for culturing solid mucoprotein is prepared as follows:

Reagents preparation:

Acid chemical compounds, including 1.491 g of FeCL₂·4H₂O, 0.06 g of H₃BO₄, 0.068 g of ZnCL₂, 0.1725 g of CuCL₂·7H₂O, 0.0635 g of MnCL₂, 0.0119 g of CoCL₂·6H₂O, 0.0235 g of NiCL₂·6H₂O, 4.18 ml of 347% HCL, and the rest being distilled water to make up a constant volume of 100 ml.

Basic chemical compounds, including 0.01729 g of Na₂SeO₃, 0.0242 g of Na₂MoO₄·2H₂O, 0.4 g of NaOH, and the rest being distilled water to make up a constant volume of 100 ml.

Liquid vitamin, including 0.02 g of biotin, 0.21 g of Vit B₃, 0.5 g of Vit B₆, 0.1 g of Vit B₂, 0.2 g of Vit B₁, 0.25 g of Vit B₁₂, 0.1 g of pantothenic acid, and the rest being distilled water to make up a constant volume of 100 ml.

Solution 1, including 1.1 g of CaCL₂, 1.0 g of MgCL₂, 1 ml of the acid chemical compounds, 1 ml of the basic chemical compounds, and the rest being distilled water to make up a constant volume of 100 ml; said solution 1 is kept at high pressure and 125° C. for 30 mins, and is subsequently kept room temperature in a sealed condition.

Solution 2, including 5.3 g of NaHPO₄, 3 g of NaCL, 4 g of KH₂PO₄, and the rest being distilled water to make up a constant volume of 100 ml; said solution 2 is kept at high pressure and 125° C. for 30 mins, and is subsequently kept at room temperature in a sealed condition.

Solution 3, including 2 g of NaHCO₃, 2 ml of the liquid vitamin, constant volume being adjusted to 100 ml, filtered by a 0.22 μm filter, and is subsequently kept at 4° C. in a sealed condition.

Solution 4, including 2.5 g of Na₂S and, the rest being distilled water to make up a constant volume of 100 ml, filtered by a 0.22 μm filter, and is subsequently kept at 4° C. in a sealed condition.

Adding 2 g of agar (sold by OXIOD, United Kingdom) to 200 ml of distilled water in a beaker, keeping the beaker at high pressure under 125° C. for 30 mins, after that immediately adding 2 ml of solution 1, 2 ml of solution 2, 1 ml of solution 3, 2 ml of solution 4 and 30-40 ml of purified mucoprotein solution sequentially to the beaker on a clean bench to form a liquid mixture, wherein the beaker is shaken evenly after each adding step, pouring the liquid mixture into a plurality of pasteurized trays each having a capacity of 20 ml, after cooling and solidifying processes, wrapping and sealing each tray in a pasteurized bag, and keeping the trays at 4° C. in a sealed condition.

The invention has the following advantageous effects over the prior art: Practical, simple, useful and convenient. The present invention makes use of the common PCR method and repeats three times the filtering of selected bases for culturing. Eventually, probiotic colonies are directly identifiable on chocolate tablets for culturing and subsequently isolated therefrom.

### BRIEF DESCRIPTION OF THE DRAWINGS

In order to explain more clearly the technical solutions proposed by the embodiment of the present invention or the technical solutions of the prior arts, the drawings necessary to illustrate the prior art or the embodiment of the present invention will be briefly described below. Obviously, the accompanying drawings are illustrative of an embodiment of the present invention. A person skilled in this field of art may obtain other drawings based on the drawings disclosed below on condition that no inventive effort will have to be made.

FIG. 1 plots light absorbance versus wavelength for mucoprotein before purification and after purification in a method of isolating probiotics of human intestine according to the invention;

FIG. 2 is a photograph showing liquid of the base for culturing liquid mucoprotein according, to the invention;

FIG. 3 shows two photographs of a base for culturing solid mucoprotein according to the invention;

FIG. 4 is a photograph of a result of gel imaging of PCR (polymerase chain reaction) products after PCR identification according to the invention;

FIG. 5 is a photograph of colonies on a chocolate tablet according to the invention; and

FIG. 6 is a photograph of colonies cultured on a Columbia blood tablet according to the invention.

### DETAILED DESCRIPTION OF THE INVENTION

An embodiment of the present invention will be fully and clearly described below with reference to the figures. Obviously, the embodiment disclosed below only one of many possible embodiments. Any other embodiments not described herein but conceivable by a person skilled in this field of art in accordance with the teachings of the present invention and without any inventive effort should also fall within the scope of protection of the present invention.

A method of isolating probiotics of human intestine in accordance with the invention is illustrated. The method comprises the following steps:

Step 1 is preparation of culture bases, including preparing a base for culturing liquid mucoprotein and preparing a base for culturing solid mucoprotein, in which mucoprotein of the bases for culturing liquid mucoprotein and solid mucoprotein is purified mucoprotein. The purification of the mucoprotein comprises the following steps:

Reagents preparation:

Ethanol without water (cooled at 4° c.);

Solution A: 0.2 g of NaH₂PO₄ without water, 7 g of Na₂HPO₄·12H₂O, 6 g of NaCL, pH adjusted to 7.8, constant volume being adjusted to 1,000 ml, kept at high pressure and 125° C. for 30 mins, and subsequently kept at room temperature

Solution B: 5.85 g of NaCL, pH adjusted to 7.0, tent volume being adjusted to 1,000 ml, and kept at high pressure and 125° C. for 30 mins; and subsequently kept at room temperature.

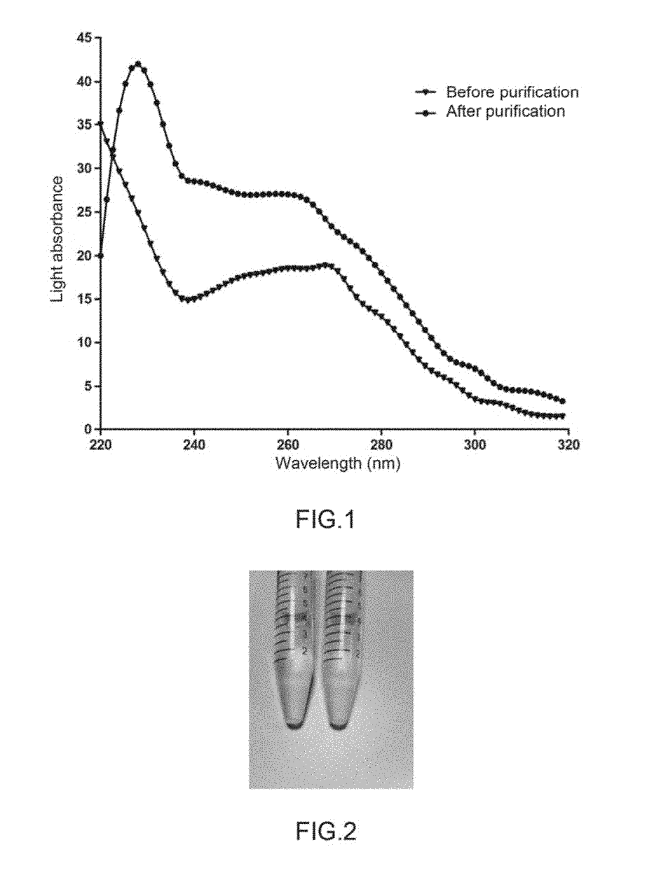

10-15 g of mucoprotein powder is added to a reagent bottle containing 500 ml of solution A to form a solution. The solution is agitated evenly by a magnetic agitator for 2 hours. 1M sodium hydroxide is added to the solution to adjust its pH value to 7.2±0.2 500-1,000 μl of toluene is added to the solution. The solution is then evenly agitated again by the magnetic agitator for 18 hours. Next, the solution is rotated by a centrifugal device rotating at 3,000 rpm for 10 mins. Liquid upper portion of the solution is collected and transferred to a second reagent bottle which has been subjected to high pressure, and deposited residue of the solution is discarded. Cooled ethanol without water is added to the liquid upper portion in the second reagent bottle to reach a concentration percentage of about 60%. The second reagent bottle placed in a refrigerator and kept at 4° C. for 30 mins. The second reagent bottle is rotated by the centrifugal device rotating at 3,000 rpm for 10 mins to obtain a second liquid upper portion and a second deposited residue. The second liquid upper portion is discarded and the second deposited residue is collected. The second deposited residue is dissolved 200 ml of solution B, and then agitated for 6 hours and after that rotated by the centrifugal device rotating at 3,000 rpm for 10 mins to obtain a third liquid upper portion and a third deposited residue. The third liquid upper portion is transferred to a third reagent bottle which has been subjected to high pressure, and the third deposited residue is discarded. Cooled ethanol without water is added to the third liquid upper portion in the third reagent bottle to reach a concentration percentage of about 60%. The third reagent bottle is placed in a refrigerator and kept at 4° C. for 30 mins. After that, the third reagent bottle is rotated by the centrifugal device rotating at 3,000 rpm for 10 mins to obtain a fourth liquid upper portion and a fourth deposited residue. The fourth liquid upper portion is discarded and the fourth deposited residue is collected. The fourth deposited residue is dissolved in 100 ml of distilled water and kept in a sealed condition at 4° C. The purified mucoprotein is free of insolvable impurities and has an increased concentration. Curve representing the purified mucoprotein has a salt peak at A230 as shown in FIG. 1.

Step 2 is filtering, including preparing a base for culturing liquid mucoprotein and preparing a base for culturing solid mucoprotein.

Reagents preparation:

Acid chemical compounds, including 1.491 g of FeCL₂·4H₂O, 0.06 g of H₃BO₄, 0.068 g of ZnCL₂, 0.1725 g of CuCL₂·7H₂O, 0.0635 g of MnCL₂, 0.0119 g of CoCL₂·6H₂O, 0.0235 g of NiCL₂·6H₂O, 4.18 ml of HCL, and the rest being distilled water to make up a constant volume of 100 ml.

Basic chemical compounds, including 0.01729 g of Na₂SeO₃, 0.0242 g of Na₂MoO₄·2H₂O, 0.4 g of NaOH, and the rest being distilled water to make up a constant volume of 100 ml.

Liquid vitamin, including 0.02 g of biotin, 0.21 g of Vit B₃, 0.5 g of Vit B₆, 0.1 g of Vit B₂, 0.2 g of Vit B₁, 0.25 g of Vit B₁₂ 0.1 g of pantothenic acid, and the rest being distilled water to make up a constant volume of 100 ml.

Solution 1, including 1.1 g of CaCL₂, 1.0 g of MgCL₂, 1 ml of the acid chemical compounds, 1 ml of the basic chemical compounds, and the rest being distilled water to make up a constant volume of 100 ml; said solution 1 is kept at high pressure and 125° C. for 30 mins, and is subsequently kept at room temperature in a sealed condition.

Solution 2, including 5.3 g of Na₂HPO₄, 3 g of NaCL, 4 g of KH₂PO₄, and the rest being distilled water to make up a constant volume of 100 ml; said solution 2 is kept at high pressure and 125° C. for 30 mins, and is subsequently kept at room temperature in a sealed condition.

Solution 3, including 2 g of NaHCO₃, 2 ml of tie liquid vitamin, constant volume being adjusted to 100 ml, filtered by a 0.22 μm filter, and is subsequently kept at 4° C. in a sealed condition.

Solution 4, including 2.5 g of Na₂S and the rest being distilled water to make up a constant volume of 100 ml, filtered by a 0.22 μm filter, and is subsequently kept at 4° C. in a sealed condition.

Preparation of a base for culturing liquid mucoprotein, including adding 200 ml of sterilized distilled water to a beaker that has been subjected to high pressure, sequentially adding 2 ml of solution 1, 2 ml of solution 2, 1 ml of solution 3, 2 ml of solution 4 and 30-40 ml of purified mucoprotein solution to the beaker on a clean bench to form a liquid mixture, wherein the beaker is shaken evenly after each adding step, sucking 5 ml of the liquid mixture of by means of an electric liquid suction device and adding 5 ml of the liquid mixture to each of a plurality of tubes having 15 ml volume, sealing the tubes, and keeping the tubes at 4° C. in a sealed condition tree FIG. 2).

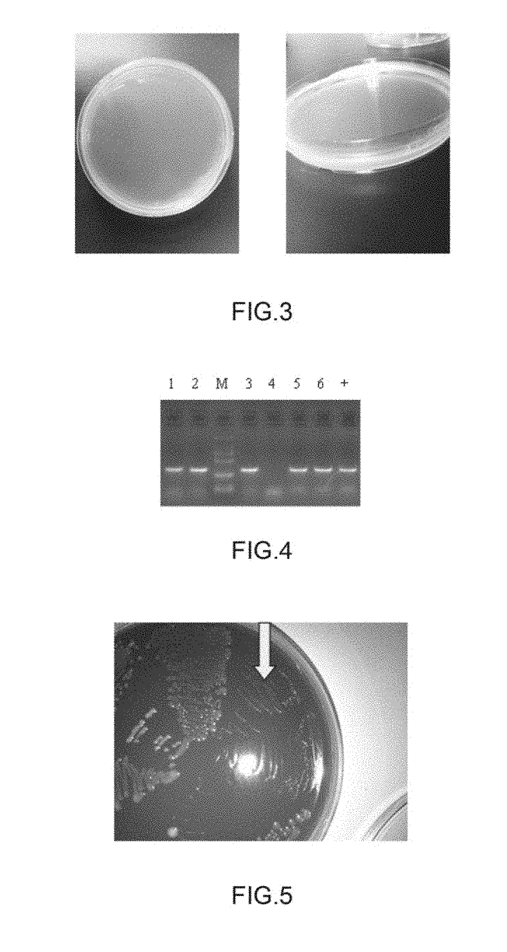

Preparation of a base for culturing solid mucoprotein, including adding 2 g of agar (sold by OXIOD, United Kingdom) to 200 ml of distilled water in a beaker, keeping the beaker at high pressure under 125° C. for 30 mins, after that immediately adding 2 ml of solution 1, 2 ml of solution 2, 1 ml of solution 3, 2 ml of solution 4 and 30-40 ml of purified mucoprotein solution sequentially to the beaker on a clean bench to form a liquid mixture, wherein the beaker is shaken evenly after each adding step, pouring the liquid mixture into a plurality of pasteurized trays each having a capacity of 20 ml, after cooling and solidifying processes, wrapping and sealing each tray in a pasteurized bag, and keeping the trays at 4° C. in a sealed condition (see FIG. 3). The base for culturing solid mucoprotein should be flat, translucent and foggy, and have a thickness of 5 mm.

A) Collection of human feces, including collecting 1-2 g of normal human feces, planting the same ire the base for culturing liquid mucoprotein, diluting the human feces ten times from 10⁻¹ to 10⁻⁶, marking and labelling the base, and placing the base in an anaerobic glove box at 37° C. for four days. It is noted that a cap of the anaerobic glove box should be loosened for ventilation.

B) Identification of the selection of the base for culturing liquid mucoprotein, including:

1. Agitating liquid in the base for culturing mucoprotein evenly, sucking 200 μl of the liquid in the base for culturing liquid mucoprotein under anaerobic environment and pouring the same into a pasteurized EP tube having a capacity of 1.5 ml, and then marking and labelling the EP tube;

2. placing the EP tube in a metal bath at 100° C. for 15 mins, and treating the liquid in the base for culturing liquid mrcoprotein as a template for conducting a subsequent PCR experiment;

3. preparing a PCR reaction solution based on the following concentration:

Special primer 1, having the sequence of SEQ ID NO: 1

(CAGCACGTGAAGGTGGGGAC);

Special primer 2, having the sequence of SEQ ID NO: 2

(CCTTGCGGTTGGCTTCAGAT)

| | |
|---|---|
| Premix Taq (TaKaRa Taq ™ | 25 μl |
| Version 2.0 plus dye) | |
| Primer 1 | 2 μl |
| Primer 2 | 2 μl |
| DD H2O | 19 μl |
| Template | 2 μl |

4. PCR reaction is conducted based on the following conditions:

| | | | |
|---|---|---|---|
| 94° C. | 2 min | | |
| 94° C. | 30 s | | |
| | | {close oversize brace} | 40 cycles |
| 60° C. | 30 s | | |
| 72° C. | 30 s | | |
| 72° C. | 5 min | | |
| 4° C. | +∞ | | |

Gel imaging of PCR product:

Absorbing 10 μl of the PCR product for electrophoresis at 135V for 35 mins. If there is an obvious strip at about 300 bp (see FIG. 4) the PCR product is positive, in which ATCC BAA-835 standard rains are used.

C) Planting on mucoprotein tablets, including:

1. Selecting tubes tested positive in PCR, discarding other tubes tested negative in PCR, and conducting further tests on any tubes tested positive and having the greatest degree of dilution;

2. Shaking evenly the tubes obtained in the previous step in an anaerobic environment, transferring the liquid n the tubes via an inoculation loop onto a plurality of mucoprotein tablets for planting, each mucoprotein tablet being sectioned to 6 to 8 zones by continuous streak plate method; and

3. Culturing the mucoprotein tablets at 37° C. in an anaerobic environment for three days.

D) Planting on chocolate tablets, including:

1. picking a loop of colonies out of the mucoprotein tablets and planting the same on the chocolate tablets in which each chocolate tablet has four zones sectioned by continuous streak plate method;

2. Culturing the chocolate tablet at 37° C. in anaerobic environment for two days; and selecting smaller colonies dispersed among the colonies out of the chocolate tablets and planting the smaller colonies on a plurality of Columbia blood tablets, as illustrated in FIG. 5. The selected smaller colonies are smaller colonies existing among bigger and et colonies or are independently existing white colonies which are wet and having circular and convex shapes well as smooth edges, as indicated by the arrow in FIG. 5.

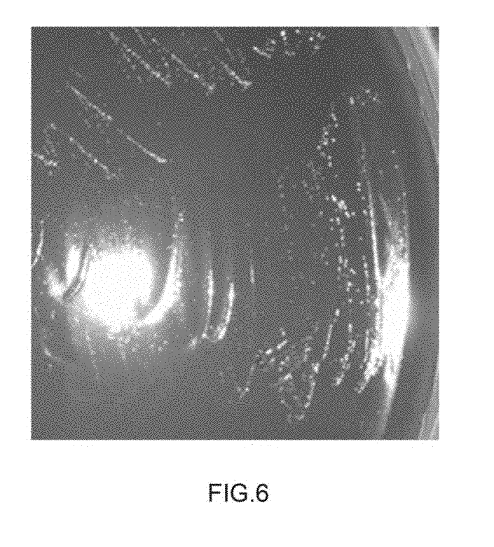

E) Purification on the Columbia blood tablets, including firstly selecting a single colony and performing purification culturing of the same on the Columbia blood tablets; and secondly, after five days, again selecting a single colony for culturing, repeating the first and second steps for three times. Finally, a purified small colonies is formed as shown in FIG. 6 having a convex and circular shape and is being white and translucent.

F) Using primer again for PCR identification, including subjecting the colonies to PCR for identification. Details are the same as described above with the template replaced by a loop of colonies.

G) Using 16sRNA common primers for sequence identification, in which primer 1 is 1492R: GGTTACCTTGTTACGACTT, and primer 2 is 27R AGAGTTTGATCCTGGCTCA.

| | |
|---|---|
| Premix Taq (TaKaRa Taq ™ | 25 μl |
| Version 2.0 plus dye) | |
| Primer 1 | 2 μl |
| Primer 2 | 2 μl |
| DD H2O | 19 μl |
| Template | a loop of colonies |

PCR is conducted based on the following conditions:

| | | | |
|---|---|---|---|
| 94° C. | 2 min | | |
| 94° C. | 30 s | | |
| | | {close oversize brace} | 40 cycles |
| 60° C. | 30 s | | |
| 72° C. | 30 s | | |
| 72° C. | 5 min | | |
| 4° C. | +∞ | | |

Gel imaging of PCR product:

Absorbing 10 μl of the PCR product for electrophoresis at 135 V for 35 mins. The PCR product is positive if there is an obvious strip at about 1500 bp. In sequence identification after Blast, it is determined whether colonies similar to probiotic C-IFH are isolated.

In subsequent implementation of the present invention, 38 C-IFH strains are isolated from feces samples of more than 200 people. The percentage of successfully isolating C-IFH strains in Chinese is about 20%.

While the invention has been described in terms of a preferred embodiment, the present invention should not be limited by the embodiment described herein. Those skilled in the art will recognize that the invention can be practiced with modifications and changes within the spirit and scope of the appended claims, and these modifications and changes should also fall within the scope of protection of the present invention.

What is claimed is:

1. A method of isolating probiotics of human intestine comprising the following steps:
Step 1: preparing culture bases, including preparing a base for culturing liquid mucoprotein and preparing a base for culturing solid mucoprotein; and
Step 2: filtering, including collecting human feces samples, planting the human feces samples in the base for culturing liquid mucoprotein, culturing the human feces samples in an anaerobic environment to obtain microbial liquid, using the microbial liquid as template for PCR test which comprises pouring the microbial liquid into tubes and subjecting the tubes to PCR test, selecting the tubes tested positive in the PCR test, planting the microbial liquid in the tubes tested positive on the base for culturing solid mucoprotein, culturing the microbial liquid on the base for culturing solid mucoprotein in an anaerobic environment to obtain first colonies, planting the first colonies in a chocolate tablet, culturing the first colonies in an anaerobic environment to obtain second colonies, planting the second colonies in a Columbia blood tablet, purifying the second colonies in an anaerobic environment to obtain third colonies, using 16sRNA common primers to identify the third colonies in PCR sequence identification, and isolating probiotics of human intestine.

2. The method of isolating probiotics of human intestine as in claim 1, wherein the mucoprotein in both the base for culturing solid mucoprotein and the base for culturing liquid mucoprotein is purified mucoprotein.

3. The method of isolating probiotics of human intestine as in claim 1, wherein the primers used in the PCR test are primer 1 having the sequence of SECS ID NO: 1 (CAGCACGTGAAGGTGGGGAC), and primer 2 having the sequence SEQ ID NO: 2 (CCTTGCGGTTGGCTTCAGAT).

4. The method of isolating probiotics of human intestine as in claim 1, wherein the human feces samples are planted on the base for culturing liquid mucoprotein for culturing in the anaerobic environment at 37° C. for four days.

5. The method of isolating probiotics of human intestine as in claim 1, wherein the microbial liquid is planted on the base for culturing solid mucoprotein for culturing in the anaerobic environment at 37° C. for three days.

6. The method of isolating probiotics of human intestine as in claim 1, wherein the first colonies are planted on the chocolate tablet for culturing in the anaerobic environment at 37° C. for two days.

7. The method of isolating probiotics of human intestine as in claim 1, wherein the planting of the second colonies in the Columbia blood tablet for purification is repeated three times, wherein in each time, purification temperature is set at 37° C. and lasts for five days.

8. The method of isolating probiotics of human intestine as in claim 2, wherein purification of the mucoprotein comprises the following steps:
Preparing reagents:
Ethanol without water (cooled at 4° C.);
Solution A: 0.2 g of NaH₂PO₄ without water, 7 g of Na₂HPO₄·12H₂O, 6 g of NaCL, pH adjusted to 7.8, constant volume being adjusted to 1,000 ml, kept at high pressure and 125° C. for 30 mins, and subsequently kept at room temperature;
Solution B: 5.85 g of NaCL, pH adjusted to 7.0, constant volume being adjusted to 1,000 ml, and kept at high pressure and 125° C. for 30 mins; and subsequently kept at room temperature;
adding 10-15 g of mucoprotein powder to a reagent bottle containing 500 ml of solution A to form a solution; agitating the solution evenly by a magnetic agitator for 2 hours; adding 1M sodium hydroxide to the solution to adjust pH value to 7.2±0.2; adding 500-1,000 μl of toluene to the solution; agitating the solution evenly by the magnetic agitator for 18 hours; subjecting the solution to rotation by a centrifugal device rotating at 3,000 rpm for 10 mins; collecting liquid upper portion of the solution and transferring the liquid upper portion of the solution to a second reagent bottle which has been subjected to high pressure, discarding deposited residue of the solution; adding cooled ethanol without water to the liquid upper portion in the second reagent bottle to reach a concentration percentage of about 60%; placing the second reagent bottle in a refrigerator and kept at 4° C. for 30 mins; subjecting the second reagent bottle to rotation by the centrifugal device rotating at 3,000 rpm for 10 mins to obtain a second liquid upper portion and a second deposited residue; discarding the second liquid upper portion and collecting the second deposited residue; dissolving the second deposited residue in 200 ml of the solution B, and then agitating for 6 hours, and after that subjecting the solution B to rotation by the centrifugal device rotating at 3,000 rpm for 10 mins to obtain a third liquid upper portion and a third deposited residue; transferring the third liquid upper portion to a third reagent bottle which has been subjected to high pressure, and discarding the third deposited residue;
adding cooled ethanol without water to the third liquid upper portion in the third reagent bottle to reach a concentration percentage of about 60%; placing the third reagent bottle in a refrigerator and keeping at 4° C. for 30 mins; subjecting the third reagent bottle to rotation by the centrifugal device rotating at 3,000 rpm for 10 mins to obtain a fourth liquid upper portion and a fourth deposited residue;
discarding the fourth liquid upper portion and collecting the fourth deposited residue; dissolving the fourth deposited residue in 100 ml of distilled water and keeping in a sealed condition at 4° C.

9. The method of isolating probiotics of human intestine as in claim 2, wherein the base for culturing liquid mucoprotein is prepared as follows:
Preparing reagents:
Acid chemical compounds, including 1.491 g of FeCL₂·4H₂O, 0.06 g of H₃BO₄, 0.068 g of ZnCL₂, 0.1725 g of CuCL₂·7H₂O, 0.0635 g of MnCL₂, 0.0119 g of CoCL₂·6H₂O, 0.0235 g of NiCL₂·6H₂O, 4.18 ml of HCL, and the rest being distilled water to make up a constant volume of 100 ml;
Basic chemical compounds, including 0.01729 g of Na₂SeO₃, 0.0242 g of Na₂MoO₄·2H₂O, 0.4 g of NaOH, and the rest being distilled water to make up a constant volume of 100 ml;
Liquid vitamin, including 0.02 g of biotin, 0.21 g of Vit B₃, 0.5 g of Vit B₂, 0.1 g of Vit B₂, 0.2 g of Bit B₁, 0.25g of Vit B₁₂, 0.1 g of pantothenic acid, and the rest being, distilled water to make up a constant volume of 100 ml;
Solution 1, including 1.1 g of CaCL₂, 1.0 g of MgCL₂, 1 ml of the acid chemical compounds, 1 ml of the basic chemical compounds, and the rest being distilled water to make up a constant volume of 100 ml; said solution 1 is kept at high pressure and 125° C. for 30 mins, and is subsequently kept at room temperature in a sealed condition;
Solution 2, including 5.3 g of Na₂HPO₄, 3 g of NaCL, 4 g of KH₂PO₄, and the rest being distilled water to make up a constant volume of 100 ml; said solution 2 is kept at high pressure and 125° C. for 30 mins, and is subsequently kept at room temperature in a sealed condition;
Solution 3, including 2 g of NaHCO₂, 2 ml of the liquid vitamin, constant volume being adjusted to 100 ml, filtered, by a 0.22 μm filter, and is subsequently kept at 4° C. in a sealed condition;
Solution 4, including 2.5 g of Na₂S and the rest being distilled water to makeup a constant volume of 100 ml, filtered by a 0.22 μm filter, and is subsequently kept at 4° C. in a sealed condition;
Adding 200 ml of sterilized distilled water to a beaker that has been subjected to high pressure, sequentially adding 2 ml of solution 1, 2 ml of solution 2, 1 ml of solution 3. 2 ml of solution 4 and 30-40 ml of purified mucoprotein solution to the beaker on a clean bench to form a liquid mixture, wherein the beaker is shaken, evenly after each adding step, sucking 5 ml of the liquid mixture by means of an electric liquid suction device and adding 5 ml of the liquid mixture to each of a plurality of tubes having 15ml volume, sealing the tubes and keeping the tubes at 4° C. in a sealed condition

10. The method of isolating probiotics of human intestine as in claim 2, wherein the base for culturing solid mucoprotein is prepared as follow:
Preparing reagents:
Acid chemical compounds, including 1.491 g of FeCL₂·4H₂O, 0.06 g of H₃BO₄, 0.068 b of ZnCL₂, 0.1725 g of CuCL₂·7H₂O, 0.0635 g of MnCL₂, 0.0119 g of CoCL₂·6H₂O, 0.0235 g of NiCL₂·6H₂O, 4.18 ml of HCL, and the rest being distilled water to make up a constant volume of 100 ml;
Basic chemical compounds, including 0.017299 g of Na₂SeO₃, 0.242 g of Na₂MoO₄·2H₂O, 0.4 g of NaOH, and the rest being distilled water to make up a constant volume of 100 ml;
Liquid vitamin, including 0.02 g of biotin, 0.21 g of Vit B₃, 0.5 g of Vit B₆, 0.1 g of Vit B₂, 0.2 g of Vit B₁, 0.5 g of Vit B₁₂, 0.1 g of pantothenic acid, and the rest being distilled water to make up a constant volume of 100 ml;
Solution 1, including 1.1 g of CaCL₂, 1.0 g of MgCL₂, 1 ml of the acid chemical compounds, 1 ml of the basic chemical compounds, and the rest being distilled water to make up a constant volume of 100 ml; said solution 1 is kept at high pressure and 125° C. for 30 mins and is subsequently kept at room temperature in a sealed condition.
Solution 2, including 5.3 g of Na₂HPO₄, 3 g of NaCL, 4 g of KH₂PO₄, and the rest being distilled water to make up a constant volume of 100 ml; said solution 2 is kept at high pressure and 125° C. for 30 mins, and is subsequently kept at room temperature in a sealed condition;
Solution 3, including 2 g of NaHCO₃, 2 ml of the liquid vitamin constant volume being adjusted to 100 ml, filtered by a 0.22 μm filter, and is subsequently kept at 4° C. in a sealed condition;
Solution 4, including 2.5 g of Na₂S and the rest being distilled water to make up a constant volume of 100 ml filtered by a 0.22 μm filter, and is subsequently kept at 4° C. in a sealed condition;
Adding 2 g of agar (sold by OXIOD, United Kingdom) to 200 ml of distilled water in a beaker, keeping the beaker at high pressure under 125° C. for 30 mins, after that immediately adding 2 ml of solution 1, 2 ml of solution 2, 1 ml of solution 3, 2 ml of solution 4 and 30-40 ml of purified mucoprotein solution sequentially to the beaker on a clean bench to form a liquid mixture, wherein the beaker is shaken evenly after each adding step, pouring the liquid mixture into a plurality of pasteurized trays each having a capacity of 20 ml, after cooling and solidifying processes, wrapping and sealing each tray in a pasteurized bag, and keeping the trays at 4 ° C. in a sealed condition.
